(19) United States
(12) Patent Application Publication
(10) Pub. No.: US 2009/0299189 A1
(43) Pub. Date: Dec. 3, 2009

(54) DUAL-FREQUENCY ULTRASOUND IMAGING OF CONTRAST AGENTS

(75) Inventors: Chih-Kuang YEH, Hsinchu (TW); Shin-Yuan SU, Hsinchu (TW)

Correspondence Address: LOWE HAUPTMAN HAM & BERNER, LLP, 1700 DIAGONAL ROAD, SUITE 300, ALEXANDRIA, VA, 22314, US

(73) Assignee: National Tsing Hua University, Hsinchu (TW)

(21) Appl. No.: 12/128,997

(22) Filed: May 29, 2008

(51) Int. Cl. A61B 8/00

(52) U.S. Cl. 600/458

(57) ABSTRACT

A method and system for imaging a tissue having contrast agents dispersed therein by exciting nonlinear response of the contrast agents using dual-frequency ultrasound, including transmitting a dual-frequency ultrasound to a target zone having the contrast agents dispersed therein, both frequencies of the dual-frequency ultrasound being higher than a resonance frequency of the contrast agents, and a frequency difference between the frequencies of the dual-frequency ultrasound is within a predetermined range surrounding the resonance frequency of the contrast agents.

### TECHNICAL FIELD

The disclosure is related to a system and method for ultrasonic imaging using dual-frequency ultrasound.

### BACKGROUND

Ultrasonic diagnostic imaging systems are capable of imaging and measuring the physiology within a body in a noninvasive manner. Materials known as contrast agents are introduced into the body to enhance ultrasonic diagnosis. The contrast agents comprise microbubbles.

Traditional ultrasound contrast agents comprise a shell made of protein or lecithoprotein and a core constituted by inert gas in a form, such as, a microbubble. An agent particle is about 1-6 micrometers (μm) in diameter and has a resonance frequency of about 2-5 Megahertz (MHz). Since the acoustic impedance of the agents differs from the acoustic impedance of tissue, the ultrasound contrast agents are used to enhance the contrast of ultrasonic diagnosis. In clinical use, the agents are broadly used in detecting the distribution pattern of capillary vessels and providing important diagnostic information to doctors based on echoes generated by the impact of ultrasound with agents.

However, the echoes usually comprise noise of backscattered wave from the human tissue background, which results in a blurred image and unclearness. Thus, without any necessary signal processing, quantitative analysis of the blood current and capillary vessel will be impossible. In order to overcome the above defects, a commercialized diagnostic system receives nonlinear echoes generated by the agents, filtering the backscattered signals and preserving the desired frequency and imaging. Generally speaking, the agents resonating at their resonance frequency generate the strongest harmonic signal.

Nevertheless, the generation of nonlinear contrast signals depends highly on the resonance between microbubbles and the incident acoustic wave insonation. Since the resonance frequencies of microbubbles of some of the commercially available contrast agents are relatively low, and some microbubbles are too large to resonate with high-frequency ultrasound, only a subpopulation of microbubbles, i.e., those having relatively smaller size, can respond to the high frequency impinging ultrasound and be excited to emit nonlinear echoes. Thus, performance and sensitivity of high-frequency imaging are limited.

Existing methods use second-harmonic imaging and sub-harmonic imaging. The shortcoming of the second-harmonic imaging is that the ultrasound is attenuated such that the depth that the second-harmonic signal can reach is limited, and high energy ultrasound transducer and high frequency detecting equipment are required. As for sub-harmonic imaging, although the penetrability is outstanding, the imaging resolution is not accurate enough to detect micro-tissue and can only be used for large regional tissue imaging. This is because generating the sub-harmonic signal generally requires a long incident pulse and a high acoustic pressure, which results in degraded resolution and the hazard of destruction of the microbubbles.

Existing imaging methods also include using amplitude modulated ultrasound to alleviate attenuation. Those methods require an additional low frequency ultrasound transducer or the use of an expensive annular transducer as a signal source.

### SUMMARY

According to at least one embodiment of the present invention, a method for inducing nonlinear scattering echoes is described. The method comprises transmitting a dual-frequency ultrasound to a target zone having contrast agents dispersed therein. Both frequencies of the dual-frequency ultrasound are greater than a resonance frequency of the contrast agents and a frequency difference between the frequencies of the dual-frequency ultrasound is within a predetermined range surrounding the resonance frequency of the contrast agents.

According to another embodiment of the present invention, a computer-readable medium comprising a set of machine-executable instructions for execution by a computer is described. Execution of the instructions causes the computer to: cause an emitter to transmit a dual-frequency ultrasound having two frequencies each greater than a resonance frequency of contrast agents to a target zone having the contrast agents dispersed therein; wherein the dual-frequency ultrasound has a frequency difference being within a predetermined range surrounding the resonance frequency of the contrast agent.

According to yet another embodiment of the present invention, a method for imaging a tissue having contrast agents dispersed therein is described. The method comprises: receiving scattering echoes from contrast agents as a result of a transmitted dual-frequency ultrasound having a frequency difference within a predetermined range surrounding the resonance frequency of the contrast agents; and imaging a pattern of the contrast agents dispersed in the tissue based on the received scattering echoes.

According to yet another embodiment of the present invention, a computer-readable medium comprising at least one set of machine-executable instructions in machine-readable form is described. Execution of the instructions by a computer causes the computer to: control an emitter for generating a dual-frequency ultrasound having two frequencies that together define a central frequency and a frequency difference, and transmitting the dual-frequency ultrasound to a tissue having contrast agents dispersed therein, wherein the frequency difference is within a predetermined range surrounding the resonance frequency of the contrast agents; control a receiver for receiving nonlinear scattering echoes from the contrast agents; and control an output for imaging and displaying a pattern of the contrast agents dispersed in the tissue based on the received scattering echoes.

According to yet another embodiment of the present invention, a system for imaging a tissue having contrast agents dispersed therein is described. The system comprises: an emitter configured to transmit a dual-frequency ultrasound to the tissue, wherein both frequencies of the dual-frequency ultrasound are greater than the resonance frequency of the contrast agents and have a frequency difference being within a predetermined range surrounding the resonance frequency of the contrast agents; a receiver configured to receive nonlinear scattering echoes from the contrast agents and transform the echoes to digital signals indicating a distribution of the contrast agents within the tissue.

According to yet another embodiment of the present invention, a system for imaging a tissue having contrast agents dispersed therein based on received echoes resulting from a transmitted dual-frequency ultrasound is described. The system comprises: a receiving transducer configured to receive nonlinear echoes from the contrast agents and transform received echoes to electric signals; a filter configured to filter the electric signals received from the receiving transducer; a pulse receiver configured to transform the filtered signals to digital data; an oscilloscope configured to provide video signals indicating a distribution of the contrast agents within the tissue based on the digital data; a computer configured to regulate the positioning of the receiving transducer and output an image of the distribution of the contrast agents within the tissue.

### BRIEF DESCRIPTION OF THE DRAWINGS

Embodiments of the present invention are illustrated by way of example, and not by limitation, in the figures of the accompanying drawings, wherein elements having the same reference numeral designations represent like elements throughout and wherein:

FIG. 1(a) is an illustration of the waveform of a dual-frequency pulse signal according to an embodiment of the present invention;

FIG. 1(b) is an illustration of the microbubble amplitude in response to the dual-frequency pulse signal shown in FIG. 1(a);

FIG. 1(c) is an illustration of the microbubble radius as a function of time, in response to the dual-frequency pulse signal shown in FIG. 1(a);

FIG. 1(d) is an illustration of the spectral band of the echoes from the microbubbles, in response to the dual-frequency pulse signal shown in FIG. 1(a);

FIG. 2 is an illustration of amplitude difference between microbubbles and de-ionized water, in response to the ultrasound having a frequency being close to the resonance frequency of the microbubbles;

FIG. 3(a) is a schematic illustration of an ultrasound contrast agents imaging system according to an embodiment of the present invention;

FIGS. 3(b), 3(c) and 3(d) are illustrations of received spectra from de-ionized water in response to the dual-frequency signals having different envelope components;

FIGS. 3(e), 3(f) and 3(g) are illustrations of received spectra from contrast agents in response to the dual-frequency signals having different envelope components;

FIG. 5(a) is an illustration of spectra band from the de-ionized water in response to the dual-frequency signal according to an embodiment of the present invention;

FIG. 5(b) is an illustration of spectra band from the contrast agents in response to the dual-frequency signal according to an embodiment of the present invention;

FIGS. 6(a)-6(c) is an illustration of amplitude difference of the second-harmonic components of the echoes, in response to the dual-frequency signals having different envelop frequencies;

FIGS. 6(d)-6(f) is an illustration of amplitude difference of the fourth-harmonic components of the echoes, in response to the dual-frequency signals having different envelop frequencies;

FIG. 7 is a schematic illustration of an ultrasound contrast agents imaging system according to an embodiment of the present invention;

FIGS. 8(a)-8(i) are illustrations of B-mode image of the contrast agents excited by the dual-frequency ultrasounds according to an embodiment of the present invention;

FIGS. 9(a) and 9(b) are illustrations of Contrast-to-Tissue ratios (CTRs) of the second-harmonic and fourth-harmonic signal received from the contrast agents excited by the dual-frequency ultrasounds according to an embodiment of the present invention;

FIGS. 10(a) -10(h) are illustrations of amplitude difference between echoes from de-ionized water and echoes from contrast agents, when excited by different envelope components;

FIG. 11 is a schematic illustration of a computer system for use in conjunction with an embodiment of the present invention; and

FIG. 12 is a schematic illustration of a flow chart according to a method of an embodiment of the present invention.

### DETAILED DESCRIPTION OF THE DRAWINGS

A method for imaging using ultrasound contrast agents and dual-frequency ultrasound, as well as a system for generating and detecting the distribution of ultrasound contrast agents within tissue, are described. In the following description, for purposes of explanation, numerous specific details are set forth in order to provide a thorough understanding of embodiments of the present invention. It will be apparent, however, that embodiments of the present invention may be practiced without these specific details.

An embodiment of the present invention uses ultrasonic dual-frequency signals generated by a signal transducer to induce nonlinear scattering echoes from ultrasound contrast agents. The dual-frequency ultrasound comprises a pulse x(t), which is constituted by a pair of high-frequency tone-bursts having different frequencies (e.g., with angular frequencies of ω₁ and ω₂, respectively). The waveform of the dual-frequency pulse signal is given by:

x(t)=cos(ω₁t)+cos(ω₂t). (1)

According to the sum-to-product formula in trigonometry, Eq. (1) can be rewritten as:

$$x ( t ) = 2 cos ( ( \frac{{ω}_{1} - {ω}_{2}}{2} ) t ) \cdot cos ( ( \frac{{ω}_{1} + {ω}_{2}}{2} ) t ) . \qquad ( 2 )$$

The Eq. (2), x(t) is viewed as an amplitude modulated pulse, wherein the carrier frequency is

$$( \frac{{ω}_{1} + {ω}_{2}}{2} )$$

(i.e., half of the sum frequency) and the envelope frequency is

$$( \frac{{ω}_{1} - {ω}_{2}}{2} )$$

(i.e., half of the difference frequency). Note that, when the carrier frequency is far beyond the envelope frequency, the modulation of carrier amplitude is similar for both the positive and negative half-cycles of the envelope signal, resulting in a higher envelope frequency. Specifically, since the carrier component in Eq. (2) alternates between negative and positive values many times during each half-cycle of the envelope component, the amplitude maxima are created in both negative and positive half-cycles of the envelope, resulting in a doubled overall envelope frequency. Consequently, the dual-frequency pulse signal has a rectified cosine envelope at the full (rather than half) difference frequency of (ω₁−ω₂). In this embodiment, such an envelope of the dual-frequency pulse signal at the difference frequency (ω₁−ω₂) serves as the excitation source of microbubbles.

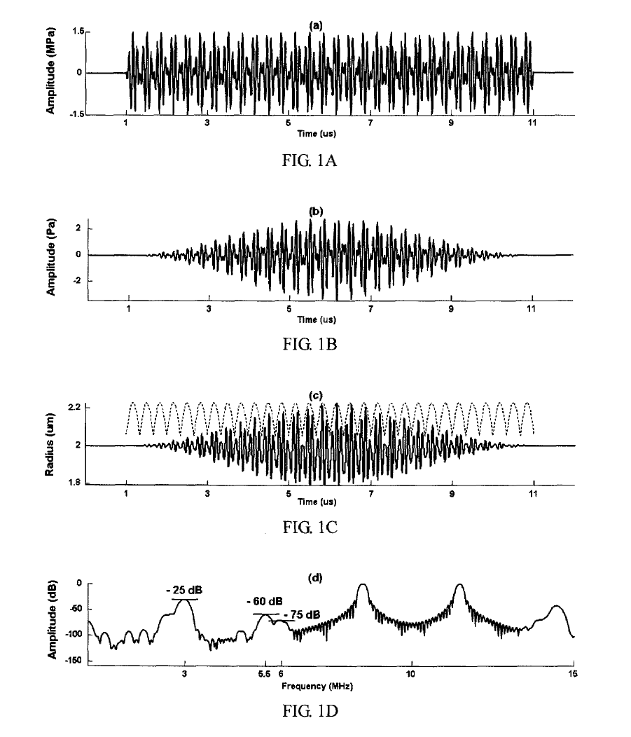

FIG. 1(a) is an illustration of a pulse signal used in the dual-frequency imaging method according to an embodiment of the present application. The pulse signal is the combination of 8.5 and 11.5 MHz tone-burst pulses. According to the above discussion, the pulse signal of FIG. 1(a) should have a carrier frequency of half of the sum frequency, i.e., (11.5+8.5)/2=10 Mhz, and an envelope frequency of the full difference frequency, i.e., (11.5−8.5)=3 Mhz. It is shown in FIG. 1(a) that the amplitude of the 10-MHz carrier waveform is modulated by a 3-MHz envelope component. This low frequency envelope is selected to be able to excite microbubbles of the particular type used in this embodiment. For other types of microbubbles, other envelope frequencies can be chosen based on a predetermined range surrounding the resonance frequency of the corresponding microbubbles herein. In this experiment as shown in FIG. 1, ultrasound contrast agents having resonance frequency of about 2 to 3 MHz (e.g., a SonoVue® microbubble available from Bracco Diagnostics, Inc, Milan, Italy) are used., and a low-frequency envelope component of 3 MHz, which is derived from the summation of the two high-frequency tone bursts (11.5 and 8.5 MHz, respectively), can be used to excite SonoVue® microbubbles. For certain types of commercial microbubbles with resonance frequencies as high as 6 MHz, the envelope frequency should be adjusted to excite the microbubbles. Especially, when the envelope frequency is within a predetermined range surrounding the microbubbles' resonance frequency, e.g., a range of about 70%-130% of the microbubbles' resonance frequency, the nonlinear oscillation from these microbubbles can be enhanced. Accordingly, the sensitivity of contrast detection can be significantly improved, while the most important benefit of high-frequency ultrasound, i.e., fine spatial resolution, can be obtained because the carrier component is still at sufficiently high frequency.

When the energy of an incident band varies, a radiation force is thereby generated. A radiation force generated by a plane wave is given as:

\vec{F}=\vec{d}ᵣS [symbol] E [symbol] (3)

where S indicates the projection area of the plane wave on the object; [symbol] E [symbol] indicates average energy density; d̅ᵣ indicates vector drag coefficient, which is defined as average incident energy density per unit projection area. As for a plane wave, its energy density is proportional to the sound field of the plane wave p(t), threshold velocity ρ and density c, and can be represented as E=p²(t)/ρc. If the sound field is given by the dual-frequency pulse signal as defined in Equation (1), then, the radiation force of the target area is given as:

F_{Δω}=P₀²dᵣS cos(Δω)/4ρc², Δω=ω₁−ω₂ (4)
P₀—pressure of incident pulse;
   According to the above equation, it appears that when dual-frequency ultrasound is transmitted to an area containing ultrasound contrast agents, the low frequency envelope (e.g., 3 MHz) of the dual-frequency ultrasound can be used to induce vibrations of the agents with a radiation force as given in Equation (4). The intensity of the force is proportional to the square of the incident sound pressure. The modulated frequency (ω₁−ω₂)/2 will generate a radiation force of a frequency of ω₁−ω₂. Because the resonance frequency of traditional ultrasound contrast agents ranges within several Mega Hz, e.g., generally between 2 and 6 MHz, mostly between 2 and 3 MHz, this embodiment of the present invention employs dual-frequency ultrasound having a frequency difference Δω of 1-3 MHz and a carrier frequency of 10 MHz to excite the agents and induce nonlinear scattering echoes from the agents. The SNR (signal/noise ratio) of the echoes can be effectively enhanced by adjusting the frequency difference and make it close to the resonate frequency of the agents.

In nonlinear microbubble scattering, received echo y(t) can be modeled as:

y(t)=a₁x(t)+a₂x²(t)+a₃x³(t)+ . . . aₙxⁿ(t), (5)

where y(t) is the backscattered echo signal from microbubbles; x(t) represents the linear component of the transmit pulse, and xⁿ(t) corresponds to the n-order nonlinear response. The contributions of the nonlinear components are determined by the coefficients aₙ. If the dual-frequency difference signal as presented in Eq. (1) is taken into account, the second-, third- and fourth-order nonlinear response are derived as in Eqs. (6), (7) and (8), respectively.

$$\begin{matrix}{x}^{2} ( t ) = 1 + \frac{1}{2} cos ( 2 {ω}_{1} t ) + \frac{1}{2} cos ( 2 {ω}_{2} t ) + cos ( ( {ω}_{1} + {ω}_{2} ) t ) + cos ( ( {ω}_{1} - {ω}_{2} ) t ) . & ( 6 ) \\ {x}^{3} ( t ) = \frac{9}{4} cos ( {ω}_{1} t ) + \frac{9}{4} cos ( {ω}_{2} t ) + \frac{1}{4} cos ( 3 {ω}_{1} t ) + \frac{1}{4} cos ( 3 {ω}_{2} t ) + \frac{3}{4} cos ( ( 2 {ω}_{2} + {ω}_{1} ) t ) + \frac{3}{4} cos ( ( {ω}_{2} + ( {ω}_{2} - {ω}_{1} ) ) t ) + \frac{3}{4} cos ( ( 2 {ω}_{1} + {ω}_{2} ) t ) + \frac{3}{4} cos ( ( {ω}_{1} + ( {ω}_{1} - {ω}_{2} ) ) t ) . & ( 7 ) \\ {x}^{4} ( t ) = \frac{9}{4} + \frac{1}{8} cos ( 4 {ω}_{1} t ) + \frac{1}{8} cos ( 4 {ω}_{2} t ) + 2 cos ( 2 {ω}_{1} t ) + 2 cos ( 2 {ω}_{2} t ) + \frac{1}{2} cos ( {ω}_{2} t - 3 {ω}_{1} t ) + \frac{1}{2} cos ( {ω}_{2} t + 3 {ω}_{1} t ) + \frac{1}{2} cos ( - ω t + 3 {ω}_{2} t ) + \frac{1}{2} cos ( {ω}_{1} t + 3 {ω}_{2} t ) + 3 cos ( ( {ω}_{1} - {ω}_{2} ) t ) + 3 cos ( ( {ω}_{1} + {ω}_{2} ) t ) + \frac{3}{4} cos ( 2 ( {ω}_{1} + {ω}_{2} ) t ) + \frac{3}{4} cos ( 2 ( {ω}_{1} - {ω}_{2} ) t ) . & ( 8 )\end{matrix}$$

As presented in Eq. (6), the second order nonlinear scattering comprises a low frequency envelope component (ω₁−ω₂), which is identical to the frequency of the acoustic force as given in Eq. (4). In Eq. (7), the third order nonlinear scattering comprises a component ω₂+(ω₂−ω₁). which can be considered as a second-harmonic signal ω₂−ω₁ modulated by original signal ω₂, thereby the component ω₂+(ω₂−ω₁) is considered as a third-harmonic signal. In Eq. (8), the fourth order nonlinear scattering comprises a doubled envelope frequency 2(ω₁−ω₂), which is considered a fourth-harmonic signal. Therefore, when a frequency difference of 3 MHz is employed (ω₁=11.5 MHz, ω₂=8.5 ), the fourth-harmonic signal is 6 MHZ (2(ω₁−ω₂)), and the third-harmonic signal is 5.5 MHz (ω₂+(ω₂−ω₁)). Note that the envelope frequency (ω₁−ω₂) is present in Eqs. (6)-(8), which indicates that the high-order nonlinear scattering of microbubble can be generated using high-frequency ultrasound with the envelope component at low frequency.

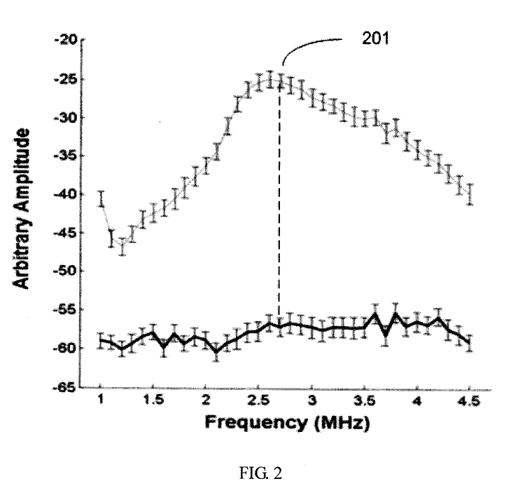

In one of the embodiments of the present invention, the dual-frequency excitation is performed on microbubbles with 2-μm radius (e.g., a SonoVue® microbubble available from Bracco Diagnostics, Inc, Milan, Italy). The resonance frequency of the SonoVue® microbubbles with 2-μm radius is close to 2.7 MHz. As illustrated in FIG. 2, the lower curve represents the response spectra band from de-ionized water under a series of frequencies, and the top curve represents the response spectra band from a 2-μm radius microbubble under the same frequencies. The peak value 201 in FIG. 2 indicates that the resonance frequency of the SonoVue® microbubble with 2-μm radius is close to 2.7 MHz. However, individual microbubbles may have different characteristics between each other, such as, size, shape, weight, etc. Commercially available microbubbles have variations in the resonance frequency. Therefore, according to one embodiment of the present invention, a frequency difference of the dual-frequency ultrasound should be within a predetermined range surrounding the resonance frequency of the microbubbles.

As shown in FIG. 1(a), a 10-μs dual-frequency pulse signal with 1.5 MPa peak pressure is utilized as the excitation waveform. Note that the resultant envelope frequency of the excitation waveform (i.e., 8.5 MHz plus 11.5 MHz tone bursts) is tuned to 3 MHz, which is about 110% of the resonance frequency of 2-μm SonoVue® microbubble. The instantaneous radius of the microbubble is approximated numerically by solving the Rayleigh-Plesset equation with an arbitrary impinging acoustic wave. Echoes from the bubble can be formulated from the bubble radius, wall velocity, and wall acceleration. For SonoVue® parameters, the shell thickness is 1 nanometers (nm), the shear modulus of the shell is 86.7 milliPascals (MPa), and the shell viscosity is 0.76×10⁻⁶ Pa·s.

FIG. 1(b) shows the amplitude of microbubbles' oscillation in response to the dual-frequency pulse signal of FIG. 1(a) processed by the Hann-window functions. FIG. 1(c) shows the microbubble radius as a function of time, and FIG. 1(d) shows the corresponding frequency response or echo y(t). As can be seen in FIG. 1(d), the amplitude of the frequency response under the excitation envelope frequency (i.e., 3 MHz) is only −25 dB lower than 8.5 or 11.5 MHz linear component. In addition, in FIG. 1(c), the envelope of microbubbles' time-radius curve is in phase with the original dual-frequency pulse signal (dashed line in FIG. 1(c)). FIG. 1(d) is an illustration of the spectral band of the echoes from the microbubbles in response to the dual-frequency pulse signal shown in FIG. 1(a). In FIG. 1(d), amplitude peaks appear at 3 MHz, 5.5 MHz and 6 MHz. The 3-MHz component of the echoes can be viewed as the second-harmonic of the envelope frequency of the dual-frequency pulse signal of FIG. 1(a). The 5.5 MHz component with −60 dB amplitude difference can be viewed as the third-harmonic of the envelope frequency and corresponds to the last term in Eq. (7). Similarly, the 6 MHz component of the frequency response (−75 dB in FIG. 1(d)) is referred to as the fourth-harmonic of the envelope frequency and corresponds to the last term in Eq. (8).

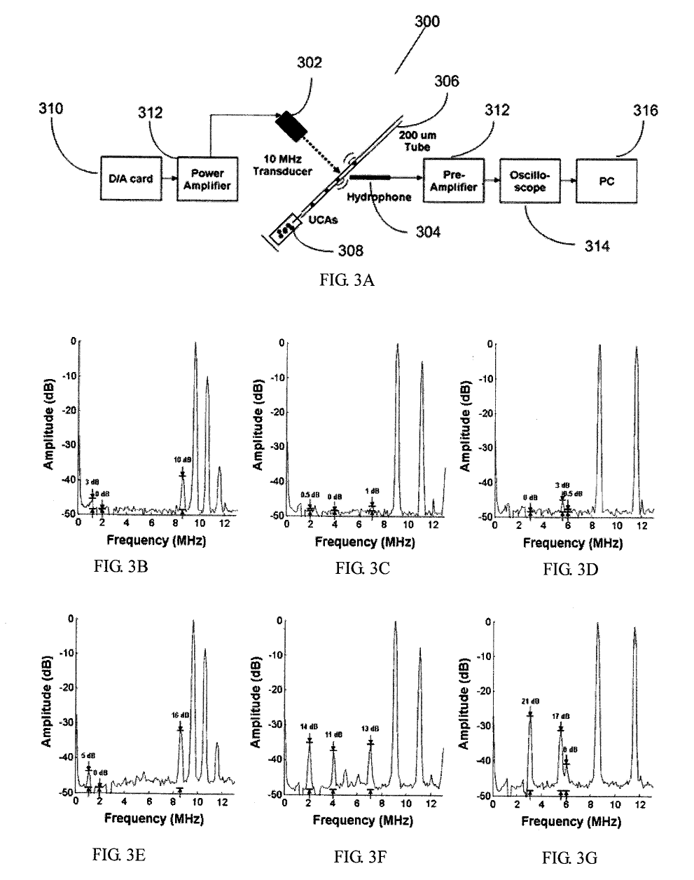

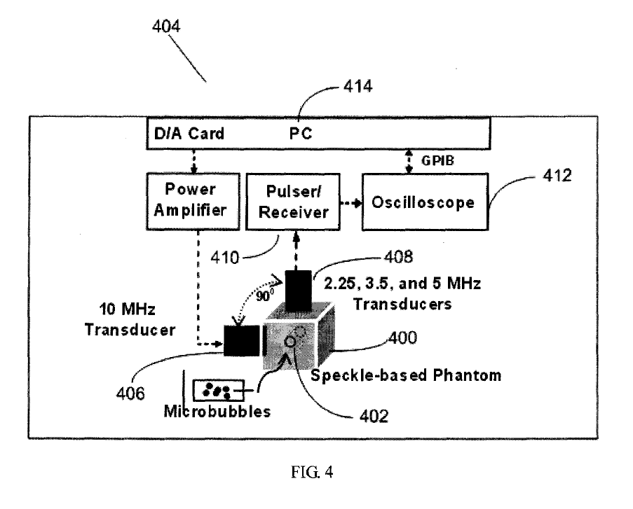

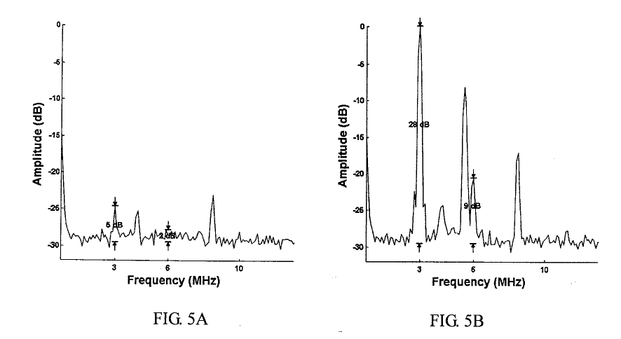

In one embodiment of the present invention, a block diagram of a measurement system 300 is shown in FIG. 3(a). The system 300 comprises transducer 302 and needle hydrophone 304. In this embodiment, the transducer 302 is a 10 MHz focused transducer (e.g., a model V322 transducer available from GE Panametrics, Waltham, Mass., USA) responsible for transmission and is fixed at a 90-degree angle with respect to a 200 μm inner diameter cellulose tube 306 (e.g., a cellulose tube available from Spectrum Labs, Laguna Hills, Calif., USA) with the focal region of transducer 302 aligned with the tube 306. Relevant parameters of transducer 302 are given in Table I.

TABLE I

| | Transducer Model | | | |
|---|---|---|---|---|
| | V322 | V305 | V381 | V308 |
| Central Frequency | 10 MHz | 2.25 MHz | 3.5 MHz | 5 MHz |
| Element Size | 25.4 | 19.1 | 19.1 | 19.1 |
| Focal Length | 50.8 | 50.8 | 50.8 | 50.8 |
| −6 dB Bandwidth | 65.0% | 72.2% | 75.9% | 58.5% |

Units: (mm)

Needle hydrophone 304 is employed (e.g., a model HNP-0400 hydrophone available from ONDA, Sunnyvale, Calif., USA) for receiving, which is fixed at a 45-degree angle with respect to tube 306 and approximately 2 millimeter (mm) away from the focal region of the transducer 302. A syringe pump (not shown) regulates the flow rate of contrast agent solution through tube 306 at 1 milliliter/hour (mL/h) (i.e., 8.9 mm/s). Contrast agents 308 may be, for example, commercial agents from SonoVue® with a concentration of 0.1 v/v %.

A digital-to-analog (D/A) card 310 (e.g., a model TE5300 D/A card available from Tabor Electronics, Tel Hanan, Israel) is used to generate the dual-frequency pulses with envelope frequencies of 1 MHz (i.e., ω₁ of 9.5 MHz and ω₂ of 10.5 MHz), 2 MHz (i.e., ω₁ of 9 MHz and ω₂ of 11 MHz) and 3 MHz (i.e., ω₁ of 8.5 MHz and ω₂ of 11.5 MHz) with 10 μs pulse length. The pulse repetition frequency (PRF) was 100 Hz. A radio frequency (RF) power amplifier 312 (e.g., a model 150A100B power amplifier available from AR, Souderton, Pa., USA) is employed to amplify the dual-frequency pulses to produce the corresponding acoustic pressure of 3.5 MPa. The RF signals received by hydrophone 304 are amplified by preamplifier 312 (e.g., an A17 dB amplifier available from ONDA, Sunnyvale, Calif., USA) and then are digitized at 100 MSamples/s using 8-bit digital oscilloscope 314 (e.g., a model LT-322 oscilloscope available from LeCroy Corporation, Chestnut Ridge, N.Y., USA). The digitized data were transferred to personal computer 316 by general purpose interface bus (GPIB) interface for analysis.

FIGS. 3(b) and (e) show the received spectra from de-ionized water and contrast agents 308 inside tube 306, respectively, in the case of 1 MHz envelope component. The amplitude differences between the spectra received from the de-ionized water and from contrast agents 308 at the frequencies of 1 MHz (i.e., second-harmonic signal), 8.5 MHz (i.e., third-harmonic signal), and 2 MHz (i.e., fourth-harmonic signal) are 2, 6, and 0 dB, respectively. FIGS. 3(c) and (f) show the results for of 2 MHz envelope component. The amplitude differences between the spectra received from the de-ionized water and from contrast agents 308 at the frequencies of 2 MHz (i.e., second-harmonic signal), 7 MHz (i.e., third-harmonic signal), and 4 MHz (i.e., fourth-harmonic) are 13.5, 12, and 11 dB, respectively. FIGS. 3(d) and (g) show the results of 3-MHz envelope component. The corresponding amplitude differences between the spectra received from the de-ionized water and from contrast agents 308 at the frequencies of 3 MHz (i.e., second-harmonic signal), 5.5 MHz (i.e., third-harmonic signal), and 6 MHz (i.e., fourth-harmonic signal) are 22, 14, and 7.5 dB, respectively. As shown in FIGS. 3(b) through 3(g), the spectral amplitudes of nonlinear components significantly increase as the envelope frequency becomes closer (i.e., 3 MHz) to the resonance frequency of SonoVue® microbubbles (i.e., 2.7 MHz).

In an experiment, human tissue background is simulated by using a speckle-based flow phantom 400, as illustrated in FIG. 4. Flow phantom 400 is made from 2 grams agarose powder in 100 mL water. Carbon powder is also uniformly included in the phantom 400 as background scatterers. A dialysis tube 402 having a diameter of 0.97 mm is embedded inside the phantom 400 and is drawn out after the agar gel congealed to form a wall-less flow channel. A measurement system 404 according to one of the embodiments of the present invention is shown in FIG. 4, which comprises a 10 MHz transducer 406 used for transmitting dual-frequency pulses, and a low-frequency focused transducer 408 for receiving echoes. Transducers 406 and 408 are positioned co-focally. Low-frequency focused transducers 408 with a different focusing frequency may be used, for example, transducers having a central frequency of 2.25, 3.5 and 5 MHz may be employed, corresponding to the envelope frequencies at 1, 2 and 3 MHz, respectively. The parameters of the transducers are also summarized in Table I. The received signals are processed by a pulser/receiver 410 (e.g., a model 5072PR pulser/receiver available from Panametric, Waltham, Mass., USA). The pulser/receiver 410 comprises a hardware filter for transforming and filtering the received passband from DC to 10 MHz. The received RF echoes are digitized at 120 Msamples/s using a computer system or controller-based 14-bit analog-to-digital board (e.g., a model PCI-9820 A/D board available from AdLink, Taipei, Taiwan) and the digital data is then transferred to oscilloscope 412 and stored on a computer system 414 for off-line processing.

FIGS. 5(a) and (b) show the frequency responses of microbubbles echoes excited by dual-frequency pulses of 3 MHz envelope frequency with 10 μs length and 1.5 MPa peak pressure as shown in FIG. 1(a). FIG. 5(a) illustrates the responses from de-ionized water and FIG. 5(b) illustrates the response from the contrast agents flowing in the flow phantom. A 5 MHz transducer is used for receiving signals. The spectral amplitudes for frequencies ranging from 15 to 20 MHz are averaged as a reference level. As for the responses from the de-ionized water, the amplitudes of the spectral peak in the frequencies of 3 and 6 MHz are 5 and 2 dB greater than the reference level, as shown in FIG. 5(a). As for the responses from the contrast agents, the amplitudes of the spectral peak in the frequencies of 3 and 6 MHz are 28 and 9 dB higher than the reference level, as shown in FIG. 5(b). The amplitude differences of the 3 and 6 MHz frequency components between FIGS. 5(a) and (b) are 23 and 7 dB, respectively. In this experiment, the responses from the de-ionized water are considered as background noise and FIGS. 5(a) and (b) illustrate that the echoes from the microbubbles are greater than the background noise.

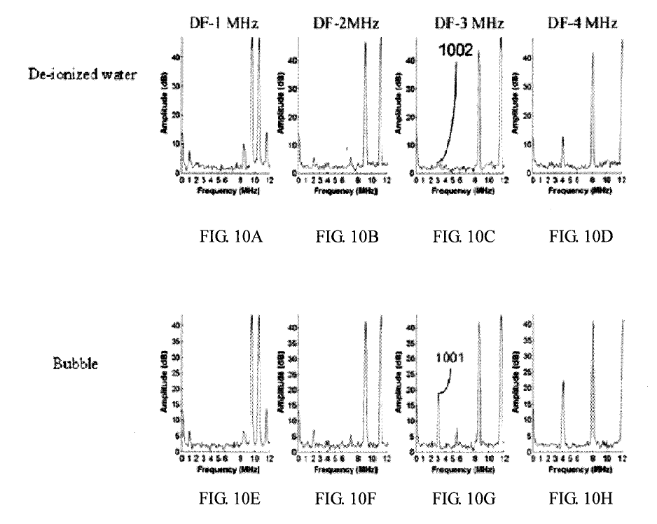

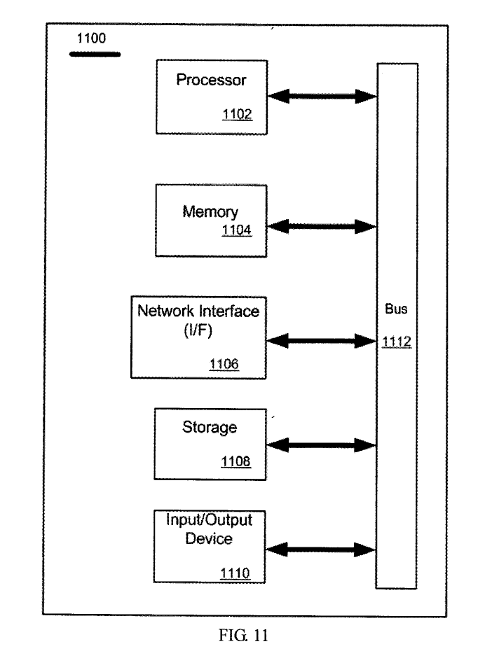

FIGS. 10(a)-(h) illustrate another experiment according to an embodiment of the present invention, wherein the contrast agents are excited under various frequency differences of dual-frequency ultrasound. In this experiment, the contrast agents are microbubbles commercially available from SonoVue® and having a resonance frequency of 2.7 MHz. FIGS. 10(a)-(d) illustrate the responses from de-ionized water under frequency differences of 1 MHz (37% of the resonance frequency of the contrast agents), 2 MHz (75% of the resonance frequency of the contrast agents), 3 MHz (110% of the resonance frequency of the contrast agents) and 4 MHz (148% of the resonance frequency of the contrast agents). FIGS. 10(e)-(h) illustrate the responses from the contrast agents flowing in the flow phantom, under the same frequency differences as FIGS. 10(a)-(d), respectively. As shown in FIGS. 10(c) and (g), when the frequency difference of the dual-frequency ultrasound is 3 MHz, the highest amplitude difference occurs, e.g., the amplitude difference between points 1001 and 1002 (point 1001 represents the amplitude of a 3 MHz component of the echoes from the microbubbles; point 1002 represents the amplitude of a 3 MHz component of the echoes from the de-ionized water). This is because the 3 MHz frequency difference (110% of the resonance frequency of the contrast agents) is the closest to the resonance frequency of the SonoVue® microbubble (2.7 MHz). In fact, in one of the embodiments of the present invention, in order to obtain an improved imaging resolution, the frequency difference of the dual-frequency ultrasound is within a predetermined range surrounding the resonance frequency of the contrast agents. In at least one embodiment, the frequency difference is configured to be identical to the resonance frequency of the contrast agents. In at least one other embodiment, the predetermined range is a range between 70-130% of the resonance frequency of the contrast agents. As shown in FIG. 10(h), the amplitude of the bubbles appears to be highest, when the frequency difference is 4 MHz. However, the amplitude difference of the nonlinear response is lower than the amplitude difference when the frequency difference is at 3 MHz. This means that the de-ionized water absorbs more energy when the frequency difference is 4 MHz, and the background noise is stronger than that of the frequency difference at 3 MHz.

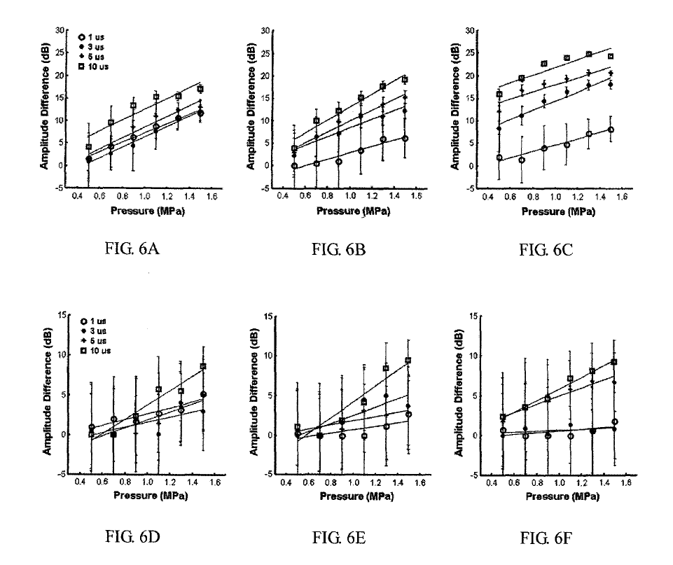

Contrast-to-noise ratios (CNRs) for six pressures ranging from 0.5 to 1.5 MPa and four pulse lengths of 1, 3, 5 and 10 μs are shown in FIG. 6. In FIGS. 6(a)-(f), 20 tests are performed. Under each pulse length of 1, 3, 5 and 10 μs, 100 tests are performed and the results are calculated to obtain mean and standard deviation of CNRs. FIGS. 6(a)-(c) represent the second-harmonic components of the nonlinear scattering excited by dual-frequency ultrasounds with envelope frequencies at 1, 2 and 3 MHz, respectively. Linear regression is performed, and the fitting lines are illustrated as solid lines. The horizontal axis represents the pressure and the vertical axis indicates the amplitude difference. The ratios of the fitting lines are greater than 0.9, which indicates that the CNRs consistently increase with pulse length and insonation pressure.

FIGS. 6(d)-(f) illustrate the CNRs of fourth-harmonic signal of the nonlinear scattering (i.e., 2, 4 and 6 MHz) excited by dual-frequency ultrasounds with envelope frequencies at 1, 2 and 3 MHz, respectively. The ratio of the fitting lines are still greater than 0.8, except in the case in FIG. 6(f). The CNR estimates in fourth-harmonics have larger variances than those in second-harmonics mainly due to their lower SNRs, it is also apparent that the case of the envelope frequency at 3 MHz generally has higher CNRs when microbubbles resonate more nonlinearly when exposed to the envelope component at or near the bubble's resonance frequency. With reference to FIGS. 5 and 6, the SNR of 3 MHz is improved with respect to the SNRs of 5.5 MHz and 6 MHz. Thus, the higher order harmonic (5.5 MHz and 6 MHz) has a larger variance, as compared to the second-harmonic signals. The results indicate that the envelope component of dual-frequency pulse signal induces significant nonlinear scattering from microbubbles.

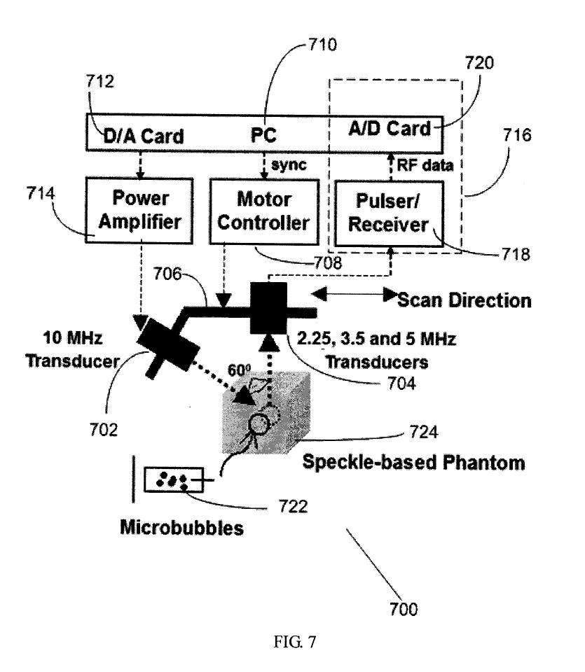

In another experiment, the phantom fabrication is similar as mentioned in the preceding section, except the vessel diameter is enlarged to 2.75 mm. A schematic diagram of the imaging system is illustrated in FIG. 7. Imaging system 700 comprises two single-element transducers: a 10 MHz transducer 702 for transmitting dual-frequency pulses, and a low-frequency transducer (hydrophone) 704 for receiving echoes. The two transducers 702 and 704 are positioned co-focally by using a holder 706, with a separation angle between the transducers of approximately 60 degrees. Transducer holder 706 is affixed to a 2-D motion stage (e.g., a model HR8 motion stage available from Nanomotion, Yokneam, Israel). Transducer 704 is actuated by a motor to be movable along transducer holder 706. The motor scanning plane is perpendicular to the flow axis of the flow phantom. A motor controller 708 (e.g., a model DMC-2140 motor controller available from Galil Motion Control, Rocklin, Calif., USA) receives instruction from computer 710 and controls the motor to position transducer 704. In operation, computer 710 controls a D/A card 712 to send electric signals representing the dual-frequency. The electric signals sent by D/A card 712 are amplified by power amplifier 714, and then sent to 10 MHz transducer 702. Echoes received by receiving transducer 704 are transferred to data acquisition system 716. Data acquisition system 716, comprising pulser/receiver 718 and A/D card 720, are controlled by software executed by computer 710. Other components of the imaging system 700 are the same as previously mentioned. A syringe pump regulates the flow rate of solution containing contrast agents 722 through the flow phantom 724 at 10 mL/h (i.e., 0.5 mm/s). The B-mode images were 4 mm in depth and 8 mm in width, and were acquired at a frame rate of approximately 1 frame per second (fps).

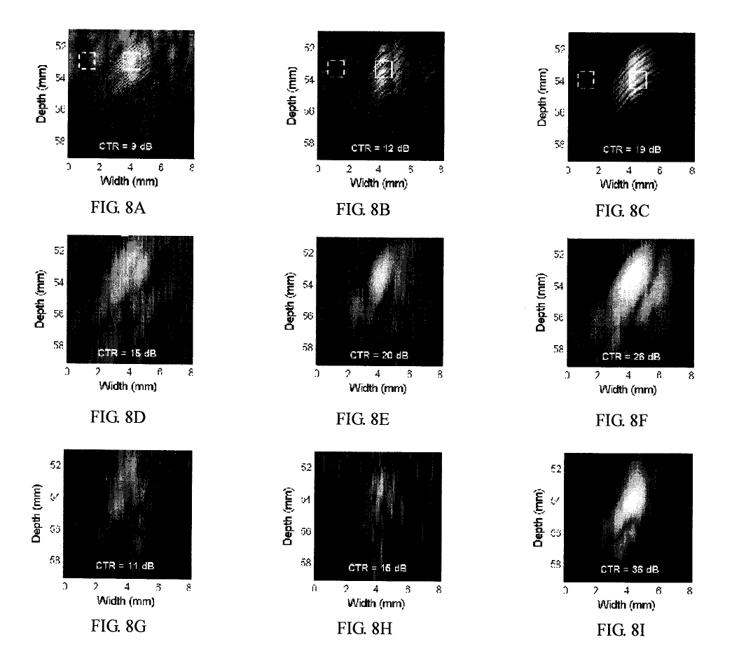

The typical B-mode images of the nonlinear echoes excited by dual-frequency pulses with envelope frequencies at 1, 2 and 3 MHz and receiving by 2.25, 3.5 and 5 MHz transducers are shown in FIGS. 8(a)-(c), respectively with the corresponding CTR values provided. The CTR is defined as the ratio of the scattered power from the contrast bubbles to the scattered power from the tissue. If the scattering signal is S, the Contrast-to-tissue ratio, CTR is given as follow:

$$CTR = 10 log [ \frac{{\sum }_{contrast} s \cdot {s}^{*}}{{\sum }_{tissue} s \cdot {s}^{*}} ] \qquad ( 9 )$$

As shown in FIG. 8(a)-(c), the CTR values derive from the brightness of two square regions with sizes of 1 by 1 mm defined by solid-line (i.e., inside the vessel) and dashed-line (i.e., background tissue), which are named as “regions of interest” (ROIs). The CTRs of the selected ROIs are 9, 12 and 19 dB, respectively. The displayed dynamic range is 40 dB for the images. The dual-frequency ultrasound pulses were 3 μs in length with a peak negative pressure of 1.5 MPa.

In this experiment, three digital filters (seventh-order band-pass Chebyshev type II digital filters with passbands of 0.85 to 1.15 MHz, 1.85 to 2.15 MHz and 2.85 to 3.15 MHz) are used to filter the original images, as illustrate FIGS. 8(a)-(c). The images of filtered second-harmonic signals are shown in FIGS. 8(d)-(f). The CTRs between the selected ROIs are 15, 20 and 26 dB, respectively. Similarly, the corresponding images of filtered fourth-harmonic signals are also shown in FIGS. 8(g)-(i), three band-pass filters (passbands from 1.85 to 2.15 MHz, 3.85 to 4.15 MHz and 5.85 to 6.15 MHz) are employed. As shown in FIGS. 8(g)-(i), the CTRs between the selected ROIs are 11, 15 and 36 dB, respectively. In FIGS. 8(d)-(i), the oblique brightness signals indicate the region of microbubbles. FIG. 8 shows that, when excited by the dual-frequency ultrasound of one or more embodiments of the present invention, the contrast microbubbles are distinguished from tissue background with sufficient image contrasts to be identified by a user or an operator, especially when only nonlinear components from bubble's oscillation is filtered for imaging. FIGS. 8(c),(f) and (i) illustrate the B-mode images of the nonlinear echoes excited by dual-frequency pulses with envelope frequency of 3 MHz and receiving by 2.25, 3.5 and 5 MHz transducers. FIGS. 8(a),(d),(g) and (b),(g),(h) illustrate the B-mode images of the nonlinear echoes excited by dual-frequency pulses with envelope frequencies of 1 and 2 MHz. Apparently, the highest resolution B-mode images occur when envelope frequency is 3 MHz, i.e., FIGS. 8(c), (f),(i), that are clearer than the images illustrated in FIGS. 8(a),(d),(g) and (b),(g),(h).

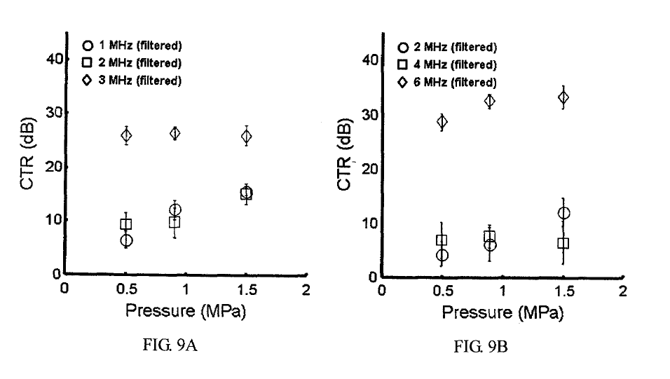

In another experiment, as illustrated in FIG. 9, the CTR values in dual-frequency ultrasound imaging are obtained under various acoustic pressures ranges from 0.5 to 1.5 MPa and different envelope frequencies of 1, 2 and 3 MHz. In each pressure level, ten independent experimental B-mode images are used to calculate the mean and standard deviation of CTRs. FIGS. 9(a) and (b) show the CTRs from images of second-harmonic signals and fourth-harmonic signals as shown in FIGS. 8(d)-(f) and FIGS. 8(g)-(i), respectively. It is also apparent in FIG. 9 that the ultrasound having an envelope component at 3 MHz provides the most pronounced nonlinear scattering from microbubbles than that of envelope components at 1 and 2 MHz. Although, the CNRs in the images of the second-harmonic signals are generally higher than those in the images of the fourth-harmonic signals because of the high noise level in fourth-harmonic signals, FIGS. 9(a) and (b) illustrate that the images of the fourth-harmonic signals provides acceptable image contrast, when the envelope frequency is at 3 MHz which is closest to the resonance frequency of microbubbles.

FIG. 11 depicts a high-level functional block diagram of system 1100, e.g., a computer system for executing a method of the present invention, according to an embodiment. System 1100 comprises a processor 1102, a memory 1104, a network interface (I/F) 1106, a storage 1108, an input/output device 1110, and a bus 1112. The processor 1102, is communicatively coupled to memory 1104, network interface (I/F) 1106, storage 1108 and input/output device 1110 through bus 1112.

Memory 1104 (also referred to as a computer-readable medium) is coupled to bus 1112 for storing data and instructions to be executed by processor 1102. Memory 1104 also may be used for storing temporary variables or other intermediate information during execution of instructions to be executed by processor 1102. Memory 1104 may also comprise a read only memory (ROM) or other static storage device coupled to bus 1112 for storing static information and instructions for processor 1102.

Network I/F 1106 comprises a mechanism for connecting to another device. In at least some embodiments, system 1100 comprises more than a single network interface.

A storage device (storage 1108), such as a magnetic disk or optical disk, may also be provided and coupled to the bus 1112 for storing data and/or instructions.

I/O device may comprise an input device, an output device and/or a combined input/output device for enabling user interaction with system 1100. An input device may comprise, for example, a keyboard, keypad, mouse, trackball, trackpad, cursor direction keys and/or an A/D card for communicating information and commands to processor 1102. An output device may comprise, for example, a display, a printer, a voice synthesizer and/or a D/A card for communicating information to a user.

The functions of a method described in connection with the embodiments disclosed herein may be embodied in hardware, executable instructions embodied in a computer-readable medium, or a combination thereof. Software comprising instructions for execution may reside in a computer-readable medium comprising volatile and/or non-volatile memory, e.g., a random access memory, a read only memory, a programmable memory, a hard disk, a compact disc, or another form of storage medium readable, directly or indirectly, by a processing device.

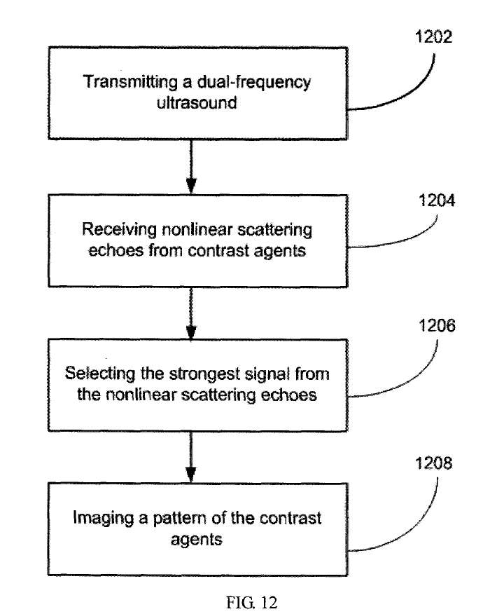

As shown in FIG. 12, according to an embodiment of the present invention, the process of imaging a tissue having contrast agents dispersed therein comprises the following steps. First, a dual-frequency ultrasound is generated and transmitted to the contrast agents, as shown in step 1202. Responsive to receipt of the dual-frequency ultrasound, the contrast agents resonate and emit nonlinear scattering echoes, and the nonlinear scattering echoes are received, as shown in step 1204. Third, the received nonlinear scattering echoes are processed, i.e., filtered and digitalized for example by system 1100, and the strongest signal are selected, as shown in step 1206. In another embodiment, the strongest signal selected may be selected after applying one or more filters to the received echoes. Finally, as shown in step 1208, a pattern image of the contrast agents dispersed in the tissue is presented in readable form and is stored in memory 1104. In at least some embodiments, the pattern image may be stored in memory 1104 and/or transmitted to another device, e.g., via network I/F 1106, in step 1208 in place of presenting in readable form.

It will be readily seen by one of ordinary skill in the art that one or more embodiments according to the present invention fulfill one or more of the objects set forth above. After reading the foregoing specification, one of ordinary skill will be able to affect various changes, substitutions of equivalents and various other embodiments of the invention as broadly disclosed herein. It is therefore intended that the protection granted hereon be limited only by the definition contained in the appended claims and equivalents thereof.

What is claimed is:

1. A method for inducing nonlinear scattering echoes, the method comprising:
transmitting a dual-frequency ultrasound to a target zone having contrast agents dispersed therein, wherein both frequencies of the dual-frequency ultrasound are greater than a resonance frequency of the contrast agents; and
wherein a frequency difference between the frequencies of the dual-frequency ultrasound is within a predetermined range surrounding the resonance frequency of the contrast agents.

2. The method of claim 1, wherein the frequency difference is equal to the resonance frequency of the contrast agents.

3. The method of claim 1, wherein the predetermined range is about 70% to 130% of the resonance frequency of the contrast agents.

4. The method of claim 1, wherein the dual-frequency ultrasound has a carrier frequency greater than the frequency difference.

5. The method of claim 4, wherein the dual-frequency ultrasound carrier frequency is at least two times greater than the frequency difference.

6. The method of claim 4, wherein the dual-frequency ultrasound carrier frequency is at least three times greater than the frequency difference.

7. A computer-readable medium comprising a set of machine-executable instructions, wherein execution of the instructions by a computer causes the computer to:
cause an emitter to transmit a dual-frequency ultrasound having two frequencies each greater than a resonance frequency of contrast agents to a target zone having the contrast agents dispersed therein; and
wherein the dual-frequency ultrasound has a frequency difference being within a predetermined range surrounding the resonance frequency of the contrast agents.

8. A method for imaging tissue having contrast agents dispersed therein, said method comprising:
receiving scattering echoes from the contrast agents as a result of a transmitted dual-frequency ultrasound having a frequency difference within a predetermined range surrounding the resonance frequency of the contrast agents; and
imaging a pattern of the contrast agents dispersed in the tissue based on the received scattering echoes.

9. The method of claim 8, further comprising:
transmitting a dual-frequency ultrasound having a frequency difference within a predetermined range surrounding the resonance frequency of the contrast agents.

10. The method of claim 8, wherein the received scattering echoes comprise nonlinear scattering echoes.

11. The method of claim 8, wherein the predetermined range is about 70% to 130% of the resonance frequency of the contrast agents.

12. The method of claim 8, wherein the dual-frequency ultrasound has two frequencies each greater than the resonance frequency of the contrast agents.

13. The method of claim 12, wherein the dual-frequency ultrasound has a carrier frequency greater than the frequency difference.

14. The method of claim 8, wherein the scattering echoes comprise at least one of a fundamental-harmonic signal, a second-harmonic signal, a third-harmonic signal or a fourth-harmonic signal of an envelope signal of the dual-frequency ultrasound.

15. The method of claim 14, further comprising selecting the strongest signal from the scattering echoes for imaging the pattern of the contrast agents.

16. A computer-readable medium comprising at least one set of machine executable instructions, wherein execution of the instructions by a computer causes the computer to:
control an emitter for generating a dual-frequency ultrasound having two frequencies that together define a central frequency and a frequency difference, and transmitting the dual-frequency ultrasound to tissue having contrast agents dispersed therein, wherein the frequency difference being within a predetermined range surrounding the resonance frequency of the contrast agents;
control a receiver for receiving nonlinear scattering echoes from the contrast agents; and
control an output for imaging and displaying a pattern of the contrast agents dispersed in the tissue based on the received nonlinear scattering echoes.

17. A system for imaging tissue having contrast agents dispersed therein, said system comprising:
an emitter configured to transmit a dual-frequency ultrasound to the tissue, wherein both frequencies of the dual-frequency ultrasound are greater than the resonance frequency of the contrast agents and wherein the frequencies have a frequency difference within a predetermined range surrounding the resonance frequency of the contrast agents;
a receiver configured to receive nonlinear scattering echoes from the contrast agents and transform the echoes to video signals indicating a distribution of the contrast agents within the tissue.

18. The system of claim 17, wherein the frequency difference is equal to the resonance frequency of the ultrasound contrast agents.

19. The system of claim 17, wherein the predetermined range is about 70%-130% of the resonance frequency of the ultrasound contrast agents.

20. The system of claim 17, wherein the central frequency of the dual-frequency ultrasound is at least 10 MHz.

21. The system of claim 17, wherein the emitter comprises:
a controller;
a digital/analog (D/A) card communicatively coupled with the controller;
a power amplifier coupled with the D/A card; and
a transducer coupled with the controller;
wherein the D/A card is configured to receive a digital signal from the controller and to transmit two analog signals to the transducer; and
wherein the transducer is configured to emit dual-frequency ultrasound to the tissue.

22. The system of claim 17, wherein the receiver further comprises:
a hydrophone configured to receive nonlinear scattering echoes from the contrast agents; and
an oscilloscope, coupled with the hydrophone, configured to provide video signals indicating a distribution of the contrast agents within the tissue.

23. The system of claim 17, further comprising a computer device configured to output an image of the distribution of the contrast agents within the tissue.

24. A system for imaging tissue having contrast agents dispersed therein, based on received echoes resulting from a transmitted dual-frequency ultrasound, said system comprising:
a receiving transducer configured to receive nonlinear echoes from the contrast agent and transform received echoes to electric signals;
a filter configured to filter the electric signals;
a pulse receiver configured to transform the filtered signals to digital data;
an oscilloscope configured to provide video signals indicating a distribution of the contrast agents within the tissue based on the digital data;
a controller configured to regulate the positioning of the receiving transducer and output an image of the distribution of the contrast agents within the tissue.

25. A method for imaging tissue having contrast agents dispersed therein, based on received echoes resulting from a transmitted dual-frequency ultrasound, said method comprising:
transforming received echoes from the contrast agents to electric signals;
filtering the received electric signals;
transforming the filtered electric signal to digital data; and
based on the digital data, generate video signals indicating a distribution of the contrast agents within the tissue.
